(19) United States
(12) Patent Application Publication
(10) Pub. No.: US 2024/0067934 A1
(43) Pub. Date: Feb. 29, 2024

(54) MODULAR VIRAL GENOMES AND METHODS OF PRODUCING THE SAME

(71) Applicant: Humane Genomics Inc., New York, NY (US)

(72) Inventors: Chad M. Moles, New York, NY (US); Peter E. Weijmarshausen, Bronxville, NY (US)

(21) Appl. No.: 18/257,544

(22) PCT Filed: Dec. 16, 2021
(86) PCT No.: PCT/US21/63930

§ 371 Date: Jun. 14, 2023

Related U.S. Application Data
(60) Provisional application No. 63/126,516, filed on Dec. 16, 2020.

(51) Int. Cl. C12N 7/00

(52) U.S. Cl. CPC C12N 7/00; C12N 2760/20222; C12N 2760/20241; C12N 2770/36122; C12N 2770/36151

(57) ABSTRACT

Disclosed herein are modular viral genomes comprising multiple individual DNA fragments, where each DNA fragment comprises a nucleic acid and at least one viral adaptor region at a terminal end of the nucleic acid. Individual DNA fragments may be assembled based on the homologous viral adaptor regions of each DNA fragment. Also disclosed herein are methods of producing a modular viral genome.

### RELATED APPLICATIONS

This application claims the benefit of U.S. Provisional Application No. 63/126,516, filed Dec. 16, 2020. The entire teachings of the above application are incorporated herein by reference.

### BACKGROUND OF THE INVENTION

According to the American Cancer Society, in 2019 there were an estimated 1,762,450 new cancer cases diagnosed and 606,880 cancer deaths in the United States. The treatment of cancer has progressed as understanding of the underlying biological processes has increased. However most current treatment options, including surgery, radiation, chemotherapy, immunotherapy, and newer targeted therapies, continue to be deployed relatively late in cancer development and have undesirable side effects even if successful in addressing the cancer. Oncolytic viral therapy (OVT), a relatively new modality that uses virus species to selectively infect, replicate, and kill cancer cells, is poised to be the next major breakthrough in the field of oncology. While this approach is promising, current methods for virus engineering are time-consuming, expensive, and require extensive scientific expertise for success. Ultimately, there is a significant unmet need for scientific approaches and biotechnologies that overcome this barrier.

### SUMMARY OF THE INVENTION

Full length nucleic acid sequences, e.g., DNA sequences, may be designed using computer aided biology (CAB), e.g., using a DNA editor or biological computer aided design (BioCAD) software. A full-length DNA is fragmented out to facilitate DNA synthesis. The DNA fragments are chemically synthesized. The DNA fragments are assembled using enzymes. The full-length physical DNA is inserted into “boot-up cells” (also referred to herein as “production cell lines” or “producer cell lines”) to make viral particles. The boot up cells may be any cell line that can be transiently or stably transfected to produce a virus of interest.

Disclosed herein are methods of producing a modular viral genome. The modular viral genome may include selecting one or more synthesized DNA fragments comprising a nucleic acid, wherein each DNA fragment comprises a first viral adaptor (VA) and a second VA, wherein the first VA is different than the second VA, and wherein each VA is located on a terminal end of the nucleic acid; and assembling the one or more synthesized DNA fragments, wherein a first synthesized DNA fragment is assembled to a second synthesized DNA fragment via homologous VA regions.

In some embodiments, the one or more synthesized DNA fragments comprise a gene. In some embodiments, the one or more synthesized DNA fragments comprise one or more genes, for example, one or more genes from a virus. In some embodiments, the virus is selected from the group consisting of measles virus (MV), rabies virus, Gibbon Ape Leukemia Virus (GALV), Sendai Virus, Seneca valley virus (SVV), adenovirus (Ad), adeno-associated viruses (AAV), herpes simplex virus (HSV), vaccinia virus (VV), vesicular stomatitis virus (VSV); autonomous parvovirus, myxoma virus (MYXV), Newcastle disease virus (NDV), reovirus, retrovirus, alphaviruses, herpesviruses, influenza virus, Sindbis virus (SINV), poxvirus, coronavirus, coronaviridae, Tobacco Mosaic Virus (TMV), Cowpea Mosaic Virus (CPMV), Semliki Forest Virus (SFV), Venezuelan equine encephalitis virus (VEEV), enterovirus, hepatitis B virus (HBV), hepatitis C virus (HCV), hepatitis D virus (HDV), and simian virus 40 (SV40).

In some embodiments, the one or more synthesized DNA fragments comprise one or more genes from one or more viruses. In some embodiments, a first synthesized DNA fragment comprises one or more genes from a first virus and a second synthesized DNA fragment comprises one or more genes from a second virus.

In some embodiments, the modular viral genome comprises at least three or, in some embodiments, at least four synthesized DNA fragments. In some embodiments, the one or more synthesized DNA fragments comprise a coding sequence (CDS), an mRNA, a 5′ untranslated region (UTR), a 3′ UTR, a signal peptide, or a mature peptide. In some embodiments, the one or more synthesized DNA fragments comprise a genetic kill switch, a reporter gene, a therapeutic agent, a watermark, a barcode, or a diagnostic agent. In some embodiments, at least one synthesized DNA fragment comprises a nucleocapsid protein (N) of VSV, a phosphoprotein (P) of VSV, a matrix protein (M) of VSV, and/or a glycoprotein (G) of VSV. In certain embodiments, at least one synthesized DNA fragment comprises a nucleocapsid protein (N), a phosphoprotein (P), and a matrix protein (M) of VSV. In some embodiments, at least one synthesized DNA fragment comprises a nonstructural protein 1 (nsP1) of SINV, a nonstructural protein 2 (nsP2) of SINV, a nonstructural protein 3 (nsP3) of SINV, a nonstructural protein 4 (nsP4) of SINV, a capsid protein (C) of SINV, a spike glycoprotein E1 (E1) of SINV, a spike glycoprotein E2 (E2) of SINV, and/or an assembly protein E3 (E3) of SINV.

Disclosed herein are methods of producing a modular viral genome. The methods may include selecting a synthesized first DNA fragment comprising a nucleic acid, a first viral adaptor (VA) and a second VA on each terminal end of the nucleic acid, wherein the first VA is different than the second VA; selecting a synthesized second DNA fragment comprising a nucleic acid, a first VA and a second VA on each terminal end of the nucleic acid, wherein the first VA is different than the second VA; and assembling the synthesized first DNA fragment and the synthesized second DNA fragment, wherein at least one VA from the synthesized first DNA fragment is homologous to at least one VA from the synthesized second DNA fragment.

In some embodiments, the methods further include selecting a synthesized third DNA fragment comprising a nucleic acid, a first VA and a second VA on each terminal end of the nucleic acid, wherein the first VA is different than the second VA, and assembling the synthesized third DNA fragment with the synthesized second DNA fragment, wherein at least one VA from the synthesized second DNA fragment is homologous to at least one VA from the synthesized third DNA fragment.

Also disclosed herein are methods of producing a modular viral genome. The methods may include designing a first DNA sequence; designing a viral adaptor (VA) region for each terminal end of the first DNA sequence; synthesizing a first DNA fragment, wherein the first DNA fragment comprises a first VA (VA1) on a first terminal end of a first nucleic acid and a second VA (VA2) on a second terminal end of the first nucleic acid; designing a second DNA sequence; designing a VA region for each terminal end of the second DNA sequence; synthesizing a second DNA fragment with a second VA (VA2) on a first terminal end of a second nucleic acid and a third VA (VA3) on a second terminal end of the second nucleic acid; and assembling the synthesized first DNA fragment and the synthesized second DNA fragment via the homologous VA2 regions of each DNA fragment to form a viral genome.

In some embodiments, the methods further include synthesizing a third DNA fragment comprising a third nucleic acid with a third VA (VA3) on a first terminal end of the third nucleic acid and a fourth VA (VA4) on a second terminal end of the third nucleic acid, and assembling the synthesized third DNA fragment to the synthesized second DNA fragment via the homologous VA3 regions.

In some embodiments, the first DNA fragment and/or the second DNA fragment each comprise a gene, or in some aspects each comprises one or more genes. In some embodiments, the first DNA fragment and/or the second DNA fragment comprises one or more genes of Vesicular stomatitis virus (VSV). In some embodiments, the first DNA fragment and/or the second DNA fragment comprises one or more genes of Sindbis virus (SINV). In some embodiments, the first DNA fragment comprises a nucleocapsid protein (N), a phosphoprotein (P), and a matrix protein (M) of VSV. In some embodiments, the second DNA fragment comprises a glycoprotein (G) of VSV. In some embodiments, the first DNA fragment and/or the second DNA fragment comprises a nonstructural protein 1 (nsP1), a nonstructural protein 2 (nsP2), a nonstructural protein 3 (nsP3), a nonstructural protein 4 (nsP4), a capsid protein (C), a spike glycoprotein E1 (E1), a spike glycoprotein E2 (E2), and an assembly protein E3 (E3) of SINV. In some embodiments, the first DNA fragment and/or the second DNA fragment comprise a coding sequence (CDS), an mRNA, a 5′ untranslated region (UTR), a 3′ UTR, a signal peptide, or a mature peptide. In some embodiments, the first DNA fragment and/or the second DNA fragment comprise a genetic kill switch, a reporter gene, a therapeutic agent, a watermark, a barcode, or a diagnostic agent.

Also disclosed herein are modular viral genomes comprising one or more synthesized DNA fragments, wherein each fragment is synthesized with a viral adaptor (VA) region on each terminal end; wherein the synthesized DNA fragments are assembled via homologous VA regions of the one or more DNA fragments.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C provide schematics of virus design. FIG. 1A shows a schematic of a virus DNA/RNA having multiple genes (G₁, G₂, G₃, Gₙ). FIG. 1B shows a schematic of a Vesicular Stomatitis Virus (VSV) RNA having 5 genes (nucleocapsid (N), phosphoprotein (P), matrix protein (M), glycoprotein (G), and large polymerase subunit (L)). FIG. 1C shows a schematic of one example of assembling a virus into three separate DNA fragments, a first DNA fragment having the N, P, M, a second DNA fragment having the G, and a third DNA fragment having the L.

FIG. 2 shows a schematic of assembling individual DNA fragments of a virus, where each DNA fragment has a homologous region (HR1, HR2, etc.). Assembly of the individual DNA fragments requires the homologous regions attached to each DNA fragment to match, e.g., an HR1 region attached to an NPM DNA fragment and an HR1 region attached to a G DNA fragment will align and allow for assembly.

FIG. 3 shows a schematic of assembling individual DNA fragments of a virus into a complete virus DNA, including inserting genes into a virus using a modular approach, as described herein. Individual DNA fragments of the virus DNA, such as NPM, G, L, as well as modular gene(s), include at least one viral adapter region (VAL VA2, VA3, etc.). The modular genes can be inserted between the individual DNA fragments by way of the viral adapter regions.

FIG. 4 shows a plasmid map of pCMV MV-H GPC3 with annotated features and enzymes.

FIG. 5 shows a schematic diagram of the DNA fragments with homologous sequences used to assemble a pCMV MV-H GPC3 plasmid.

FIG. 6 provides a photograph of a transformation plate. Following de novo DNA assembly, NEB stable cells were transformed with product and incubated at 30° C. for 24 hours. Bacteria colonies were then picked from the transformation plate, cultured, and screened for the assembled DNA.

FIG. 7 provides a photograph showing a gel analysis. To determine the size of the plasmid, DNA was extracted using commercial minipreps and analyzed by gel electrophoresis. Of the 8 colonies that were randomly selected, it was observed that 100% had the desired size plasmid.

FIG. 8 shows the DNA ladder as a reference for interpretation of the gel analysis shown in FIG. 7.

FIG. 9 provides confirmation of DNA size by nanopore sequencing using the MinION (Oxford Nanopore Technologies). Read lengths were evaluated and predominantly measured 7.44-7.55 kb long (target is 7.5 kb).

FIG. 10 demonstrates sequence verification of the plasmid. Nanopore sequencing was used to verify DNA assembly. Fastq files were aligned to a reference, the plasmid sequence of pCMV MV-H GPC3, using minimap2 alignment software. The coverage plot demonstrates that the sequence of all fragments are present.

FIG. 11 demonstrates plasmid validation. The Clone Validation workflow of EPI2ME was used to analyze the plasmid construct. A consensus sequence was determined and annotated. A graphical representation of the plasmid with annotated features was generated. The sequence length, feature identity and orientation are correctly presented, without the input of a reference file.

### DETAILED DESCRIPTION OF THE INVENTION

It is possible to chemically create synthetic or artificial pieces of deoxyribonucleic acid (“DNA”) using a process called “DNA synthesis”. These chemically created pieces of DNA, referred to herein as “DNA fragments”, can be modeled after the contents of a computer file. This enables the design of DNA on a computer, after which the DNA can be chemically made and used for various purposes. The digital representations of DNA are referred to herein as “DNA sequences”. In the context of this application, the chemically made DNA will be used to construct viruses, referred to herein as “artificial viruses”. Currently it is possible to chemically create DNA fragments of up to about 10,000 base pairs. For reference, genomes of commonly used oncolytic viruses range from about 11,000 to about 190,000 base pairs. In a typical process the DNA fragments are synthesized first, after which the DNA fragments are combined in a vessel and, using enzymes, these fragments assemble to form a complete viral genome. The process of combining the DNA fragments into the complete viral genome is called genome assembly. For clarity, the complete viral genome is not a virus, but rather the genetic material or “biological blueprint” is inserted into so called host cell lines to create the viral particles.

In summary, the process for preparing an artificial virus may comprise designing a full-length DNA sequence on a computer using a DNA editor. The full-length DNA may then be separated into individual fragments to facilitate DNA synthesis. The DNA fragments may be chemically synthesized from the designed DNA sequence and assembled using enzymes. The full-length physical DNA, e.g., a complete viral genome, may be verified by transformation in bacteria and analysis using gel electrophoresis and/or sequencing. The verified DNA may then be inserted into boot-up cells to produce viral particles of an artificial virus.

It is of note that the DNA of natural viruses can be subdivided into multiple genes. The artificially created viruses are also composed of multiple genes as well as promoter sequences, watermarks and other non-gene specific features. The fact that the chemical synthesis is limited to DNA fragments and the DNA can be divided into individual portions leads to the idea to make the DNA fragments modular. It allows for the re-use of different DNA fragments to assemble different virus variants. The ability to modify the DNA sequences and synthesize individual DNA fragments based on the DNA sequence provides a cost effective and quick turnaround time method for producing multiple variations of an artificial virus for use.

Artificial viruses may be designed for a number of biomedical applications, such as for producing vaccines, treating cancer, genetic editing, drug delivery, antimicrobials, nanodevices, and basic research. The design and production of an artificial virus comprises multiple steps, including designing nucleic acid sequences (e.g., DNA sequences) using BioCAD software and producing synthesized nucleic acid fragments (e.g., DNA fragments) based on the designed nucleic acid sequences. In many situations it is desirable to make minor modifications to a sequence using the editing system (e.g., adding or removing genes or other functional domains, or changing the location of a gene or functional domain within a full DNA sequence). In addition, a full DNA sequence may be fragmented into individual portions prior to synthesis. In some aspects, each individual DNA sequence includes a viral adaptor (VA) region at the terminal ends of the DNA sequence. In some aspects, the viral adaptor regions aid in the assembly of synthesized DNA fragments. By providing pre-synthesized DNA fragments having viral adaptor regions at the terminal end of each DNA fragment, individual pieces of the artificial virus may be taken off the shelf and combined with other DNA fragments having homologous viral adaptor regions in a timely and efficient manner for the production of an artificial virus.

Unless defined otherwise, all technical and scientific terms used herein have the meaning commonly understood by a person skilled in the art. The following references provide one of skill with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Hale & Marham, The Harper Collins Dictionary of Biology (1991); and David M. Knipe et al., Fields Virology (6^{th} ed. 2013).

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3.sup.rd ed., Cold Spring Harbor Laboratory Press, Cold Spring Harbor, N.Y.; Jeremy W. Dale et al., 2012, From Genes to Genomes: Concepts and Applications of DNA Technology, 3^{rd} ed.; and Strachan et al., 2011, Human Molecular Genetics, 4^{th} ed., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

Disclosed herein are artificial viruses and methods of producing the same. Artificial viruses comprise one or more individual nucleic acid fragments (e.g., DNA fragments) comprising nucleic acid(s) that are assembled to form the artificial virus. Each individual DNA fragment is synthesized from a DNA sequence. In some aspects, the DNA sequence is designed using DNA editing software. It is generally understood by those of skill in the art that there are various DNA editing programs available for the design and edit of nucleic acid sequences. In one embodiment, a full DNA sequence is designed using the DNA editing software and is then fragmented into individual portions to facilitate DNA synthesis. The individual DNA fragments may be synthesized using methods known to those of skill in the art. In some aspects, each individual DNA sequence comprises at least one viral adaptor (VA) region at a terminal end of the nucleic acid sequence. In some embodiments, a DNA sequence comprises a first VA region at a first terminal end of the DNA sequence and a second, different, VA region at a second terminal end of the DNA sequence. In certain embodiments, the DNA sequence comprising the VA region(s) is synthesized into a DNA fragment comprising the VA region(s). A complete viral genome may be assembled by aligning matching or homologous VA regions of the individual DNA fragments.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e., two or more alleles exist) at a genetic locus in the individuals of a population. As used herein, “mutant” refers to a change in the sequence of a nucleic acid or its encoded protein, polypeptide, or peptide that is the result of recombinant DNA technology (also referred to herein as genetic or genome engineering).

A nucleic acid may be made by any technique known to one of ordinary skill in the art. Non-limiting examples of a synthetic nucleic acid, particularly a synthetic oligonucleotide, include a nucleic acid made by in vitro chemical synthesis using phosphotriester, phosphite or phosphoramidite chemistry and solid phase techniques such as described in EP 266,032, or via deoxynucleoside H-phosphonate intermediates as described by Froehler et al., 1986, and U.S. Pat. No. 5,705,629. Non-limiting examples of enzymatically produced nucleic acids include those produced using isothermal amplification, terminal deoxynucleotidyl transferase (TdT) (see for example, Eisenstein, “Enzymatic DNA synthesis enters new phase” Nature Biotechnology, 38, 1113-1115 (2020)), enzymes in amplification reactions such as PCR™ (see for example, U.S. Pat. Nos. 4,683,202 and 4,682,195), or the synthesis of oligonucleotides (see for example U.S. Pat. No. 5,645,897). A non-limiting example of a biologically produced nucleic acid includes recombinant nucleic acid production in living cells, such as recombinant DNA vector production in bacteria (see for example, Sambrook et al. 1989).

The nucleic acid(s), regardless of the length of the sequence itself, may be combined with other nucleic acid sequences, including but not limited to, promoters, enhancers, polyadenylation signals, restriction enzyme sites, multiple cloning sites, coding segments, ribosome binding site (RBS), genetic insulators, coding sequences, and the like, to create one or more nucleic acid construct(s) of artificial viruses. The overall length may vary considerably between nucleic acid constructs. Thus, a nucleic acid segment of almost any length may be employed, with the total length preferably being limited by the ease of preparation or use in the intended recombinant nucleic acid protocol.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as an artificial virus, a gene delivery system, or gene transfer “vehicle”) refers to a macromolecule or complex of molecules comprising a polynucleotide to be delivered to a host cell, either in vitro or in vivo. A “plasmid,” a common type of a vector, is an extra-chromosomal DNA molecule separate from the chromosomal DNA that is capable of replicating independently of the chromosomal DNA. In certain cases, it is circular and double-stranded.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. Promoter motifs may be included upstream or downstream relative to the transcription start site (TSS), these may include TATA-box, initiator, GC-box, CCAAT-box sequences, and the like.

By “operably linked” or “co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

In some embodiments, a modular viral genome or artificial virus is designed to comprises one, two, three, four, five, six, seven, eight, nine, ten, fifteen, twenty, twenty-five, thirty, forty, and fifty individual fragments of a nucleic acid, e.g., DNA or RNA fragments. In some embodiments, a modular viral genome or artificial virus comprises at least one, at least five, at least ten, at least twenty, at least thirty, or at least forty individual fragments of a nucleic acid. In some embodiments, a modular viral genome or artificial virus comprises fifty or less individual fragments of a nucleic acid. In some embodiments, a modular viral genome or artificial virus comprises one to fifty, five to forty-five, ten to forty, fifteen to thirty-five, or twenty to thirty individual fragments of a nucleic acid. In some embodiments, an artificial virus comprises one DNA fragment. In some embodiments, an artificial virus comprises two DNA fragments. In some embodiments, an artificial virus comprises three DNA fragments. In some embodiments, an artificial virus comprises four DNA fragments. In some embodiments, an artificial virus comprises five DNA fragments. In some embodiments, an artificial virus comprises six DNA fragments. In some embodiments, an artificial virus comprises seven DNA fragments. In some embodiments, an artificial virus comprises eight DNA fragments. In some embodiments, an artificial virus comprises nine DNA fragments. In some embodiments, an artificial virus comprises ten DNA fragments. In some embodiments, an artificial virus comprises fifteen DNA fragments. In some embodiments, an artificial virus comprises twenty DNA fragments. In some embodiments, an artificial virus comprises twenty-five DNA fragments. In some embodiments, an artificial virus comprises thirty DNA fragments. In some embodiments, an artificial virus comprises forty DNA fragments. In some embodiments, an artificial virus comprises fifty DNA fragments. In some embodiments, a complete viral genome is assembled comprising one or more DNA fragments.

In some embodiments, a DNA fragment is synthesized from a DNA sequence. In some embodiments, the DNA sequence is a portion of a full virus DNA sequence. In other embodiments, the DNA sequence is a full virus DNA sequence. In some embodiments, a DNA sequence comprises one or more genes, a coding sequence (CDS), mRNA, a 5′ untranslated region (UTR), a 3′ UTR, a signal peptide, a mature peptide, a genetic kill switch, a reporter gene, a therapeutic agent, a watermark, a barcode, or a diagnostic agent. In some embodiments, a DNA sequence comprises one, two, three, four, five, six, seven, eight, nine, ten, fifteen, twenty, twenty-five, thirty, forty, or fifty genes. In some embodiments, a DNA sequence comprises at least one, at least five, at least ten, at least twenty, at least thirty, or at least forty genes. In some embodiments, a DNA sequence comprises fifty or less genes. In some embodiments, a DNA sequence comprises one to fifty, five to forty five, ten to forty, fifteen to thirty five, or twenty to thirty genes. In some embodiments, a DNA sequence comprises one gene. In some embodiments, a DNA sequence comprises two genes. In some embodiments, a DNA sequence comprises three genes. In some embodiments, a DNA sequence comprises four genes. In some embodiments, a DNA sequence comprises five genes. In some embodiments, a DNA sequence comprises six genes. In some embodiments, a DNA sequence comprises seven genes. In some embodiments, a DNA sequence comprises eight genes. In some embodiments, a DNA sequence comprises nine genes. In some embodiments, a DNA sequence comprises ten genes. In some embodiments, a DNA sequence comprises fifteen genes. In some embodiments, a DNA sequence comprises twenty genes. In some embodiments, a DNA sequence comprises twenty-five genes. In some embodiments, a DNA sequence comprises thirty genes. In some embodiments, a DNA sequence comprises forty genes. In some embodiments, a DNA sequence comprises fifty genes. In some embodiments, a DNA sequence comprises a kill switch that will eliminate virus assembly, virus production, or virus replication capacity, e.g., any kill switch known to those of skill in the art, including, but not limited to, a genetic kill switch or a tet-on/off system (i.e., a system that turns on a gene that inhibits the virus or turns off an essential viral gene). In one embodiment, a kill switch comprises the insertion of hepatitis C virus protease (HCV-NS3/4A) between a glycoprotein (e.g., VSV-G) and a large protein (e.g., VSV-L) in a DNA sequence and then administering an HCV inhibitor at a later time point as needed to kill or eliminate virus assembly/production/replication capacity. In one embodiment, a kill switch comprises the insertion of a riboswitch and aptamer sequence between a large protein (e.g., VSV-L) and a trunk or terminator sequence. In some aspects, the riboswitch will automatically cleave and destabilize the viral RNA unless a target protein (e.g., a cancer specific protein) is bound to the aptamer. In some embodiments, a DNA sequence comprises a reporter gene, e.g., any reporter gene known to those of skill in the art, including, but not limited to, bioluminescent genes (e.g., Luc), fluorescent genes (e.g., RFP, GFP, BFP, DsRed, mCherry, EGFP, EBFP, TxRed, moxGFP, moxBFP, tdTomato), and genes related to clinical imaging modalities (e.g., sodium iodide symporter (NIS)).

In some embodiments, one or more DNA sequences are viral DNA sequences. Non-limiting examples of viruses that may be used in forming the one or more DNA sequences include, but are not limited to, measles virus (MV), rabies virus, Gibbon Ape Leukemia Virus (GALV), Sendai Virus, Seneca valley virus (SVV), adenovirus (Ad), adeno-associated viruses (AAV), herpes simplex virus (HSV), vaccinia virus (VV), vesicular stomatitis virus (VSV); autonomous parvovirus, myxoma virus (MYXV), Newcastle disease virus (NDV), reovirus, retrovirus, alphaviruses, herpesviruses, influenza virus, Sindbis virus (SINV), poxvirus, coronavirus, coronaviridae, Tobacco Mosaic Virus (TMV), Cowpea Mosaic Virus (CPMV), Semliki Forest Virus (SFV), Venezuelan equine encephalitis virus (VEEV), hepatitis B virus (HBV), hepatitis C virus (HCV), hepatitis D virus (HDV), or simian virus 40 (SV40). For example, in some embodiments the virus can be a member of the Rhabdoviridae family, such as from the genus Vesiculovirus. In certain instances, the virus can be Indiana vesiculovirus (VSIV) or New Jersey vesiculovirus (VSNJV). In certain instances, the virus can be Sindbis virus (SINV) or vesicular stomatitis virus (VSV).

In some embodiments, a DNA sequence, e.g., a full virus DNA sequence, is designed to comprise one or more portions of the sequence from a first virus and one or more portions of the sequence from a second virus. In some embodiments, an artificial virus comprises a first DNA fragment synthesized from a DNA sequence of a first virus and a second DNA fragment synthesized from a DNA sequence of a second virus. In some embodiments, a DNA sequence, e.g., a full virus DNA sequence, is designed to comprise a first portion of the sequence from a first virus, a second portion of the sequence from a second virus, and a third portion of the sequence from a third virus. In some aspects, an artificial virus comprises a first DNA fragment synthesized from a DNA sequence of a first virus, a second DNA fragment synthesized from a DNA sequence of a second virus, and a third DNA fragment synthesized from a DNA sequence of a third virus.

In some embodiments, a DNA sequence comprises one or more genes from a virus. In some embodiments, a DNA sequence comprises one or more structural polypeptides and/or one or more nonstructural polypeptides. In some embodiments, a DNA sequence comprises one or more genes from a vesicular stomatitis virus (VSV). In some embodiments, a DNA sequence comprises one or more genes from a Sindbis virus (SINV). In some embodiments, a DNA sequence comprises one or more genes selected from the group consisting of nucleocapsid protein (N), phosphoprotein (P), matrix protein (M), glycoprotein (G), and large polymerase subunit (L). In some embodiments, a DNA sequence comprises one or more genes selected from the group consisting of nonstructural protein 1 (nsP1), nonstructural protein 2 (nsP2), nonstructural protein 3 (nsP3), nonstructural protein 4 (nsP4), capsid protein (C), spike glycoprotein E1 (E1), spike glycoprotein E2 (E2), and assembly protein E3 (E3).

In some embodiments, a DNA sequence comprises a VSV nucleocapsid protein (N). In some embodiments, a DNA sequence comprises a VSV phosphoprotein (P). In some embodiments, a DNA sequence comprises a VSV matrix protein (M). In some embodiments, a DNA sequence comprises a VSV glycoprotein (G). In some embodiments, a DNA sequence comprises a VSV large polymerase subunit (L). In some embodiments, a DNA sequence comprises a VSV nucleocapsid protein (N), phosphoprotein (P), and matrix protein (M). In some embodiments, a DNA sequence comprises a SINV nonstructural protein 1 (nsP1). In some embodiments, a DNA sequence comprises a SINV nonstructural protein 2 (nsP2). In some embodiments, a DNA sequence comprises a SINV nonstructural protein 3 (nsP3). In some embodiments, a DNA sequence comprises a SINV nonstructural protein 4 (nsP4). In some embodiments, a DNA sequence comprises a SINV capsid protein (C). In some embodiments, a DNA sequence comprises a SINV spike glycoprotein E1 (E1). In some embodiments, a DNA sequence comprises a SINV spike glycoprotein E2 (E2). In some embodiments, a DNA sequence comprises a SINV assembly protein E3 (E3). In some embodiments, a DNA sequence comprises a SINV nonstructural protein 1 (nsP1), nonstructural protein 2 (nsP2), nonstructural protein 3 (nsP3), nonstructural protein 4 (nsP4), capsid protein (C), spike glycoprotein E1 (E1), spike glycoprotein E2 (E2), and assembly protein E3 (E3).

In some embodiments, a DNA sequence comprises a measles fusion protein (F). In some embodiments, a DNA sequence comprises a measles hemagglutinin protein (H). In some embodiments, a DNA sequence comprises a GALV envelope protein (env). In some embodiments, a DNA sequence comprises a SVV VP4, VP2, and/or VP0. In some embodiments, a DNA sequence comprises a HA Tag (Derivative of Human Influenza Hemagglutinin). In some embodiments, a DNA sequence comprises a Nipah Virus envelope protein. In some embodiments, a DNA sequence comprises a Herpes Simplex Virus glycoprotein (e.g., gD, gB, gH/gL, and/or gC). In some embodiments, a DNA sequence comprises an adenovirus fiber. In some embodiments, a DNA sequence comprises a severe acute respiratory syndrome coronavirus 2 (SARS-CoV-2) glycoprotein. In some embodiments, a DNA sequence comprises a SARS-CoV-2 nucleocapsid protein. In some embodiments, a DNA fragment is synthesized from a DNA sequence comprising one or more genes from a virus described herein.

In some embodiments, a DNA sequence comprises non-functional DNA or RNA. In some embodiments, a DNA sequence comprises at least 300 base pairs of non-functional DNA or RNA.

Each DNA sequence disclosed herein may include one viral adaptor (VA) region located at one end of the sequence. Alternatively, each DNA sequence may include two viral adaptor (VA) regions, one on each end of the sequence. Where the DNA sequence includes two VA regions, each VA region is unique. For example, in some aspects, a DNA sequence may include a first VA region (VA1) and a second VA region (VA2). In some aspects, a VA1 region may be on the 5′ end of the DNA sequence and a VA2 region may be on the 3′ end of the DNA sequence. DNA fragments may be synthesized from DNA sequences comprising one or two VA regions, such that the synthesized DNA fragment comprises a VA region on one end and/or on both ends of the nucleic acid.

In some embodiments, a viral adaptor region is engineered and designed using a digital editor. In some aspects, the viral adaptors are designed to be located at one or more terminal ends of a DNA sequence, e.g., a DNA sequence comprising a viral gene or a functional domain. The viral adaptor region is designed to be at about 20 to 80 base pairs in length, about 40 to 60 base pairs in length, or in some aspects about 42 to 57 base pairs in length. In some aspects, the viral adaptor region is designed to comprise a blunt end, and in some aspects to be a double stranded DNA. The viral adaptors may have a GC content of about 20-60%, or more preferably about 29-40%. In some embodiments, a DNA sequence is designed to comprise one or more genes, a coding sequence (CDS), mRNA, a 5′ untranslated region (UTR), a 3′ UTR, a signal peptide, a mature peptide, a kill switch, a reporter gene, a therapeutic agent, a watermark, a barcode, or a diagnostic agent, and at least one, and in certain embodiments, two viral adaptor regions. DNA fragments are synthesized from the DNA sequences to comprise the nucleic acid portion and the at least one, and in certain embodiments, two viral adapter regions, where the viral adaptor regions are located at opposite ends of the nucleic acid. The viral adaptor regions located at one end of the nucleic acid is different from the viral adaptor region located at the other end of the nucleic acid.

In some embodiments, multiple DNA fragments are synthesized, each containing the same nucleic acid, but having different VA regions. For example, a first DNA fragment comprising a modular gene, such as a kill switch, has a first VA region (VA1) and a second VA region (VA2) and a second DNA fragment comprising the same modular gene, e.g., the kill switch, has a third VA region (VA3) and a fourth VA region (VA4) (as shown below). The addition of different viral adaptor regions to a nucleic acid, such as a modular gene, allows that DNA fragment to be inserted or assembled within the full virus DNA at different locations, based on aligning the viral adaptor regions of the DNA fragments with homologous viral adaptor regions.

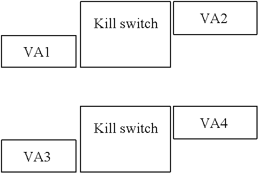

In some embodiments, multiple DNA fragments, each containing a different nucleic acid, have analogous VA regions. For example, a first DNA fragment comprising a modular gene, such as a kill switch, has a first VA region (VA1) and a second VA region (VA2) and a second DNA fragment comprising a modular gene, such as a marker, has a first VA region (VA1) and a second VA region (VA2). DNA fragments having different nucleic acids, such as different modular genes, but the same viral adaptor regions allows the DNA fragments to be swapped in and out of the same location in a complete viral genome, e.g., the kill switch may replace the marker at the same location within the viral genome based on the corresponding viral adaptor regions during assembly of the full DNA.

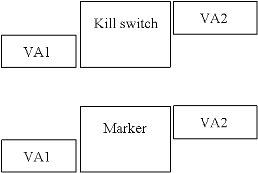

In some embodiments, a complete viral genome includes one or more DNA fragments comprising one or more genes and one or more DNA fragments comprising one or more functional domains selected from the group consisting of a genetic kill switch, a reporter gene, a therapeutic agent, a watermark, a barcode, and a diagnostic agent. In some embodiments, the complete viral genome comprises one or more DNA fragments comprising one or more genes, one or more DNA fragments comprising one or more functional domains, and one or more DNA fragments comprising non-functional DNA.

In some embodiments, a complete viral genome is obtained by selecting one or more DNA fragments described herein and assembling the one or more DNA fragments based on the viral adaptor regions included as part of each DNA fragment. For example, a first DNA fragment comprising a nucleic acid and at least one VA region, such as VA1, is assembled with a second DNA fragment comprising a nucleic acid and at least one VA region, such as VA1. The overlapping or homologous VA1 regions facilitate the assembly of the two DNA fragments.

In some embodiments, the complete viral genome is obtained by selecting one or more synthesized DNA fragments and assembling the selected DNA fragments to form a complete viral genome. In some embodiments, each DNA fragment comprises a nucleic acid and at least one viral adaptor region. In some aspects, the nucleic acid comprises one or more genes, a coding sequence (CDS), mRNA, a 5′ untranslated region (UTR), a 3′ UTR, a signal peptide, a mature peptide, a kill switch, a reporter gene, a therapeutic agent, a watermark, a barcode, or a diagnostic agent. In some aspects, each DNA fragment comprises one viral adaptor region. In other aspects, each DNA fragment comprises two viral adaptor regions, where a first viral adaptor region is located at one end of the nucleic acid, e.g., a 5′ end, and the second viral adaptor region is located at the opposing end of the nucleic acid, e.g., a 3′ end.

In one example, multiple DNA fragments are selected comprising one or more viral genes, e.g., a first DNA fragment comprises a VSV nucleocapsid protein (N), phosphoprotein (P), and matrix protein (M) segment and a second DNA fragment comprises a glycoprotein (G) segment. Each DNA fragment further comprises unique viral adaptor regions, e.g., VA1, VA2, VA3, VA4. In addition, an additional DNA fragment is selected comprising a modular gene, such as a kill switch. The additional DNA fragment comprising the modular gene further comprises viral adaptor regions that align with the viral adaptor regions of the DNA fragments comprising the one or more viral genes. This allows the modular gene, e.g., the kill switch to be inserted between the DNA fragments comprising the viral genes during assembly.

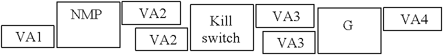

In some embodiments, a DNA fragment that includes non-functional DNA is selected for inclusion between two DNA fragments comprising one or more viral genes. By incorporating a DNA fragment that includes non-functional DNA, two DNA fragments that do not have homologous viral adaptor regions may be assembled without inserting a DNA fragment comprising a modular gene.

In some embodiments, an artificial virus is obtained by transfecting cells with a complete viral genome, where the complete viral genome is assembled from one or more DNA fragments. The modular assembly of the complete viral genome allows for the efficient production of multiple versions of an artificial virus, e.g., multiple versions of a VSV based artificial virus, where each version of the artificial virus comprises the inclusion of one or more functional domains or the modification of specific genes, e.g., replacing a VSV glycoprotein with a SARS-CoV-2 spike glycoprotein or SVV glycoprotein.

It is to be understood that the invention is not limited in its application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain compounds, compositions and methods of the present invention have been described with specificity in accordance with certain embodiments, the following examples serve only to illustrate the methods and compositions of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### EXEMPLIFICATION

It is possible to chemically create synthetic or artificial pieces of deoxyribonucleic acid (“DNA”) using a process called “DNA synthesis”. These chemically created pieces of DNA, referred to herein as “DNA fragments”, can be modeled after the contents of a computer file. This enables the design of DNA on a computer, after which the DNA can be chemically made and used for various purposes. The digital representations of DNA are referred to herein as “DNA sequences”. In the context of this application, the chemically made DNA will be used to construct viruses, referred to herein as “artificial viruses”. Currently it is possible to chemically create DNA fragments of up to about 10,000 base pairs. In a typical process the DNA fragments are synthesized first, after which the DNA fragments are combined in a vessel and, using enzymes, these fragments assemble to form a complete viral genome. The process of combining the DNA fragments into the complete viral genome is called genome assembly. For clarity, the complete viral genome is not a virus, but rather the genetic “blueprint” or material is inserted into so-called host cell lines to create the viral particles.

In summary, the process for preparing an artificial virus may comprise designing a full-length DNA sequence on a computer using a DNA editor. The full-length DNA may then be separated into individual fragments to facilitate DNA synthesis. The DNA fragments may be chemically synthesized from the designed DNA sequence and assembled using enzymes. The full-length physical DNA, e.g., a complete viral genome, may be inserted into boot-up cells to produce viral particles of an artificial virus.

It is of note that the DNA of natural viruses can be subdivided into multiple genes. The artificially created viruses are also composed of multiple genes as well as promoter sequences, watermarks and other non-gene specific pieces. The fact that the chemical synthesis is limited to DNA fragments and the DNA can be divided into individual portions leads to the idea to make the DNA fragments modular. It allows for the re-use of different DNA fragments to assemble different virus variants. The ability to modify the DNA sequences and synthesize individual DNA fragments based on the DNA sequence provides a cost effective and quick turnaround time method for producing multiple variations of an artificial virus for use.

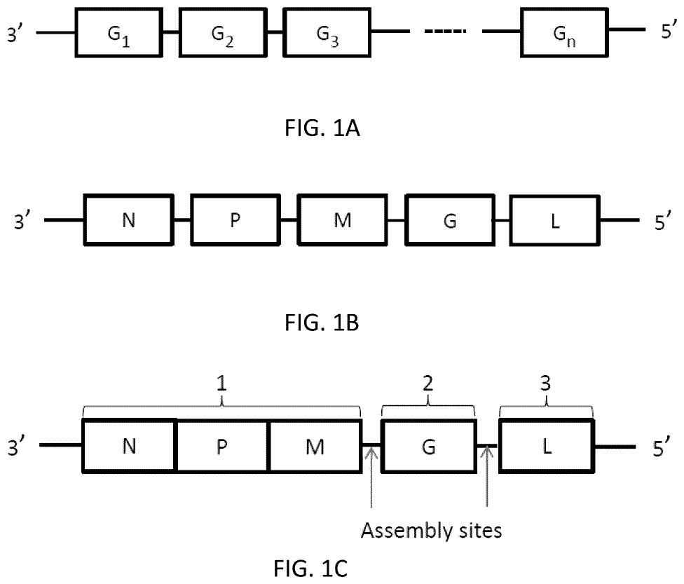

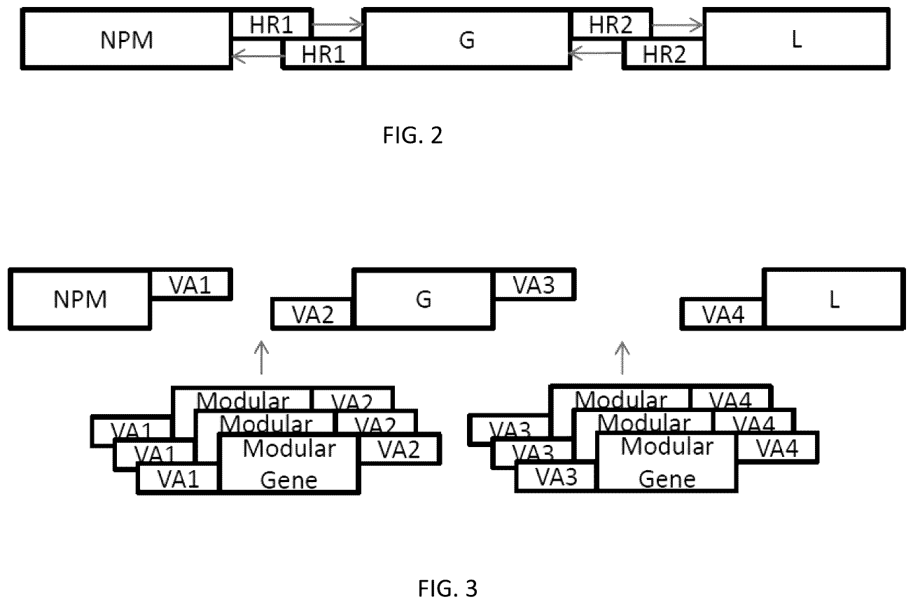

As shown in FIG. 1C, DNA fragments may comprise one or more genes. A first DNA fragment may comprise VSV nucleocapsid (N), phosphoprotein (P), and matrix protein (M) genes; a second DNA fragment may comprise the VSV glycoprotein (G) gene; and the third fragment may comprise the large polymerase protein (L) subunit. There are two identified assembly sites, one located between DNA fragment 1 and DNA fragment 2, and the second located between DNA fragment 2 and DNA fragment 3. Each assembly site comprises a homologous region (HR1, HR2) (see FIG. 2).

The assembly of the individual DNA fragments requires the presence of the homologous region (HR1 to HR1, HR2 to HR2). In this example, the first DNA fragment comprising VSV-NPM can only assemble with the second DNA fragment comprising VSV-G, and cannot assemble with the third DNA fragment comprising VSV-L because the homologous regions do not align.

Using a modular approach as described herein, the first DNA fragment comprising VSV-NPM no longer assembles with the second DNA fragment comprising VSV-G, and the second DNA fragment VSV-G no longer assembles with the third DNA fragment comprising VSV-L. Rather, additional DNA fragments comprising one or more modular genes, other functional DNA fragments, or non-functional DNA fragments are available to be inserted between the first DNA fragment and second DNA fragment and between the second DNA fragment and the third DNA fragment based on the standardized homologous regions, i.e., the viral adaptor (VA) regions (see FIG. 3).

By standardizing the homologous regions or viral adaptor regions it becomes possible to chemically synthesize unique modular pieces, i.e., DNA fragments, which can be precisely assembled into different artificial virus variants. Any combination of DNA fragments may be designed and assembled using the homologous VA regions for each DNA fragment. The benefits of this approach are that numerous artificial viruses can be designed and prepared by utilizing various combinations of DNA fragments that are pre-synthesized to include one or more viral adaptor region(s). This reduces the time to wait for chemical synthesis and it reduces the cost to make each variation.

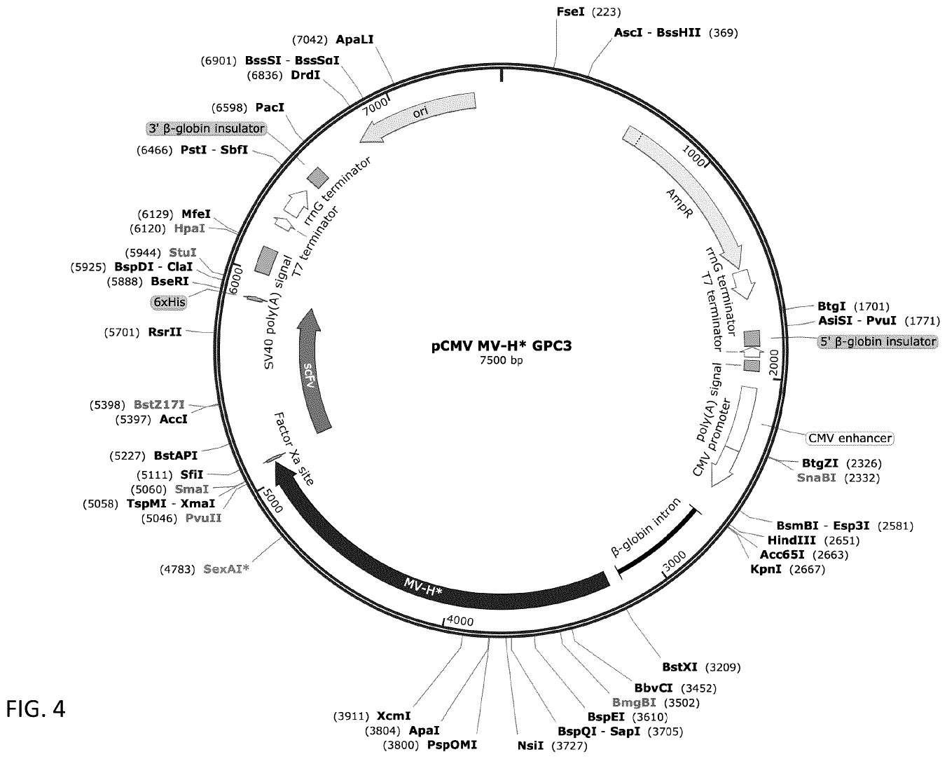

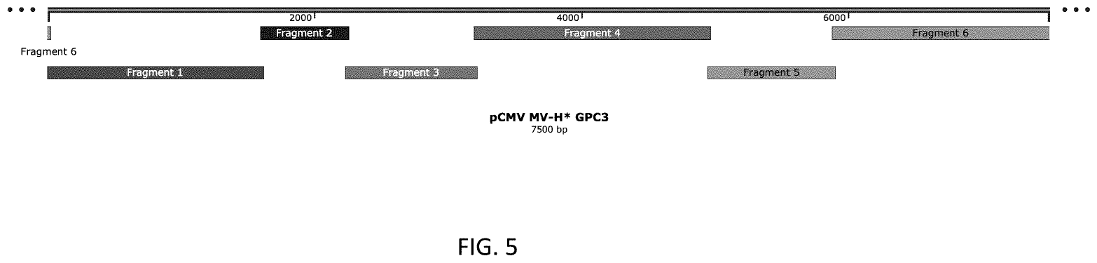

DNA fragments with homologous regions or viral adaptors may also be assembled to construct a subgenomic component or viral gene for functional validation. Measles virus hemagglutinin gene (MV-H) may be mutated to eliminate natural targeting of receptors (SLAM, CD46, and nectin-4), and then an antibody, ligand, or other targeting sequence may be added to the 3′ end for retargeting. Plasmids of glycoprotein variants may be evaluated for functionality prior to incorporating the design in a viral vector or particle. As shown in FIG. 4, MV-H is designed with mutations and a GPC3 antibody sequence fused at the 3′ end. Protein expression is driven by a CMV promoter and stabilized by a SV40 poly-A sequence. Other genes in the plasmid enable transformation and screening in bacteria. As shown in FIG. 5, the plasmid is designed as six DNA fragments with a homologous region (HR) on each end to enable assembly. Each HR is sequence specific and designed to only assemble with the adjacent fragment in the corresponding order and orientation.

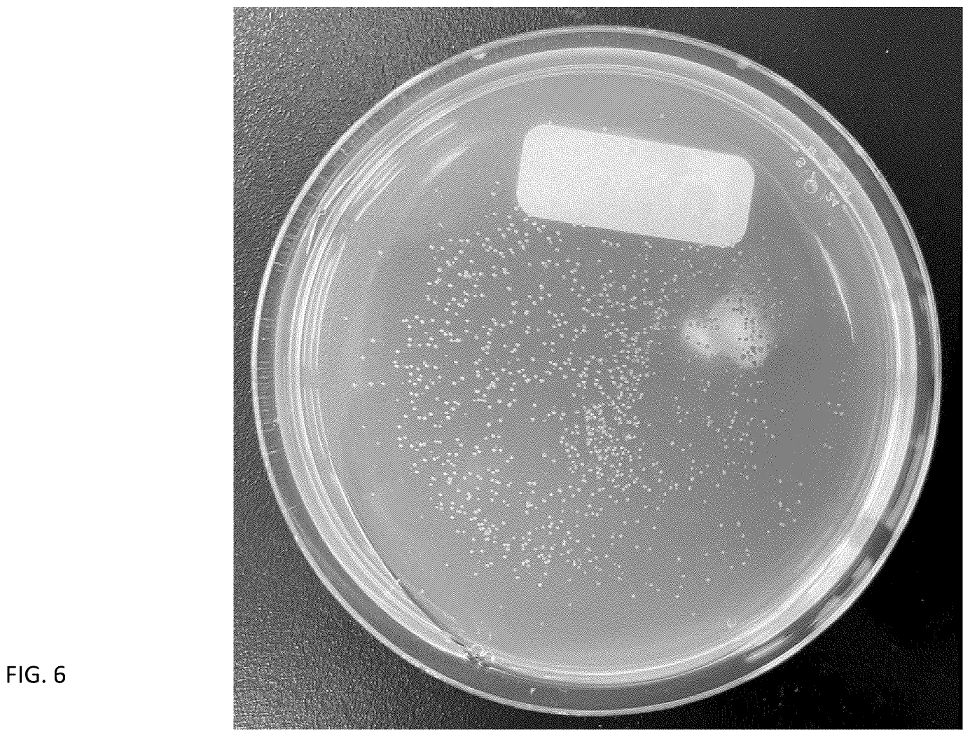

As shown in FIG. 6, the fragments were assembled and the product transformed in NEB Stable cells. E. coli contains exonucleases that degrade linear DNA, so individual fragments will be broken down. Circular DNA or plasmids may be copied and passed onto offspring. Traditional methods of viral genome engineering involve modifying existing plasmids, so transformation will yield a mixture of older template plasmid and new modified plasmid. As such, colonies must be screened for the correct plasmid. Here, the plasmids are constructed de novo, so colonies should contain only the desired plasmid. The benefits of this approach are that less colonies need to be picked and validated before proceeding, resulting in less reagents being needed for DNA purification and less time spent handling colonies.

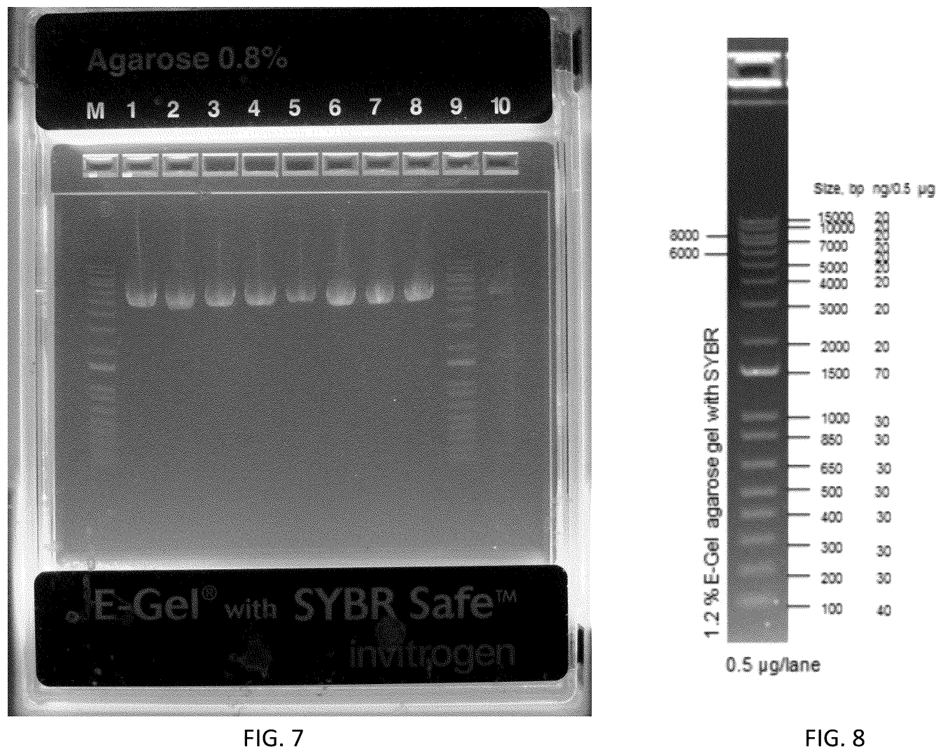

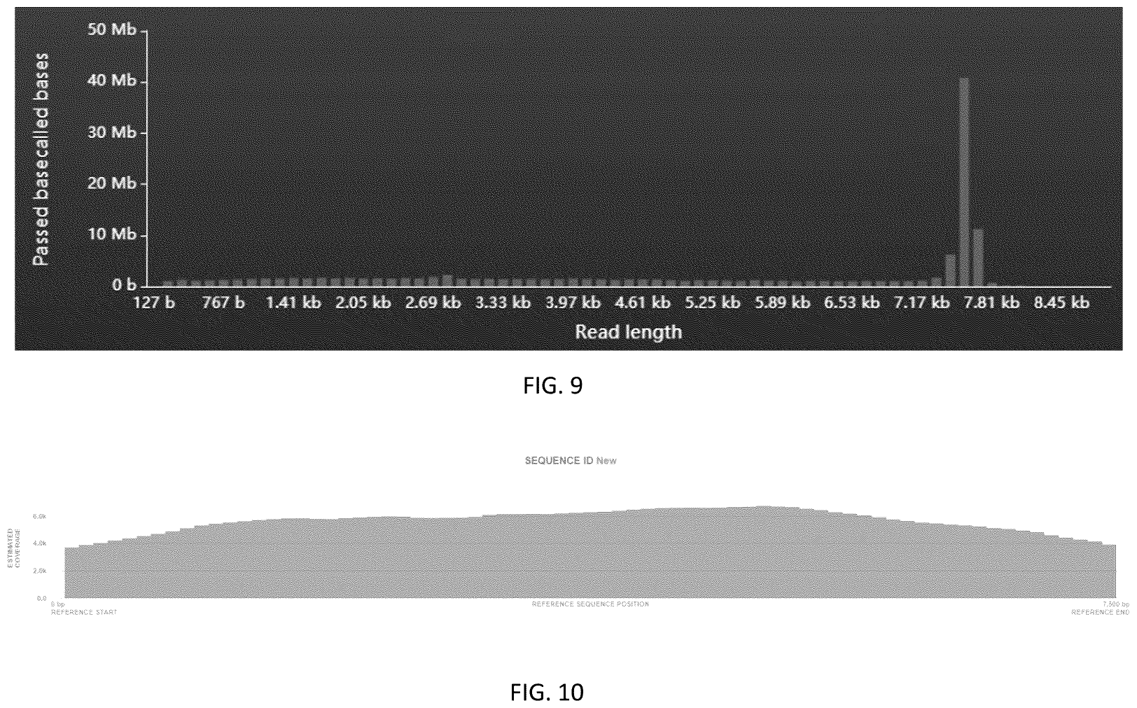

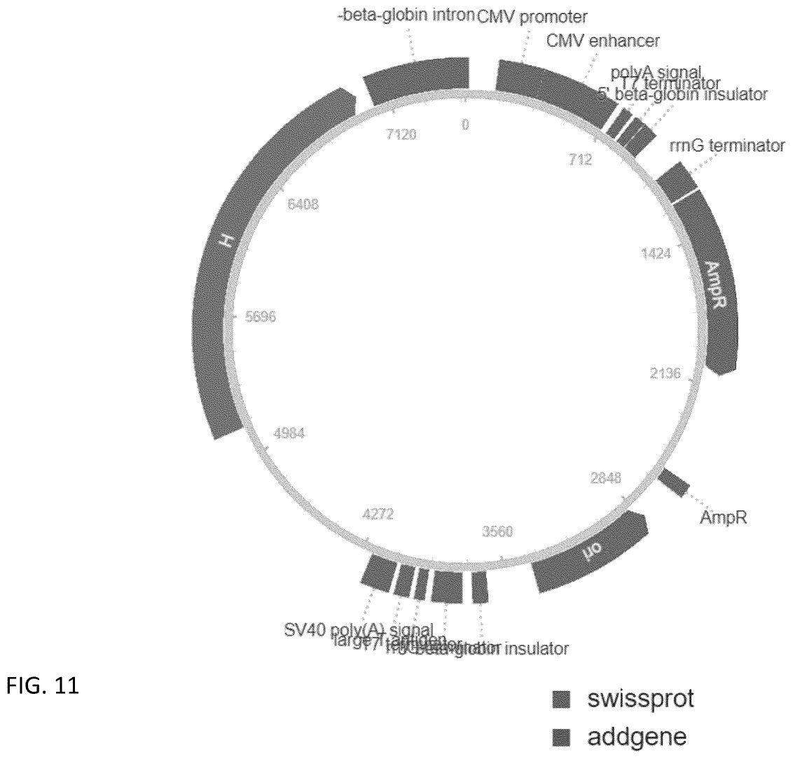

To verify that the NEB Stable colonies contained the MV-H GPC3 plasmid, DNA was extracted from eight colonies, purified, and then analyzed by gel electrophoresis and nanopore sequencing. As shown in FIG. 7, the gel showed all 8 colonies contained a similar size plasmid (all bands are aligned), and that the size was around 7,000 bp based on the reference ladder (seen to the left of the gel image and in FIG. 8). Sequence verification was performed afterwards using the MinION (Oxford Nanopore Technologies). As shown in FIG. 9, the read lengths average size was 7.44-7.55 kb, which is the target size (the plasmid is 7.5 kb). The reads were analyzed and aligned to a reference file (the MV-H GPC3 plasmid). Nanopore sequencing showed coverage of every position (FIG. 10). Furthermore, when a reference file was not provided, the nanopore software (EPI2ME) developed a consensus sequence for the input DNA and annotated it. As shown in FIG. 11, the sequence and features developed by the software are an exact match for the MV-H GPC3 plasmid. Taken together, de novo design and assembly yielded colonies that contained the assembled construct 100% of the time.

### Cell Culture

BHK-21 [C-13] (CCL-10) and 293T (CRL-3216) cell lines were purchased from ATCC. The cells were maintained in DMEM (Corning) supplemented with 10% FBS (Gibco), 5% Penicillin/Streptomycin (Corning), and 1% Amphotericin B (Corning) at 37° C. and 5% CO2.

### Viral Adaptors (VA)

Viral Adaptors were designed for the terminal ends of nucleic acids, such as VSV genes and DNA fragments. VAs facilitate DNA synthesis for complex designs and enable modular assembly of fragments. Adaptors contain 20-80 bp homologous overlap sequences with a GC content of 30-60%, which serves as a buffer between preceding poly A sequences (<35%) and subsequent transgenes (>60%), limiting overall GC % variation.

### Genome Assembly

The complete genome design was deconstructed into 0.3-7 kb fragments, which were manufactured via Twist Bioscience's silicon-based technology platform. Following DNA synthesis, 1000 ng sequence verified gene fragments were resuspended in 10 uL of TE Buffer. To construct full-length virus genomes, amplified fragments were assembled on ice via homologous recombination of viral adaptor sequences.

For HiFi DNA assembly, 0.05 pmols of DNA fragments were used with 10 uL of master mix. Nuclease-free water (NFW) was added, if needed, to bring the total reaction volume to 20 uL. The samples were then incubated at 50° C. for 60 minutes in a thermocycler. Samples were cooled to 4° C. and then used immediately or stored at −80° C.

For NUGE DNA assembly, the reaction contained 0.15 pmol of mini fragments (<3000 bp) and 0.05 pmol of mega fragments (>3000 bp) for a volume up to 5 uL. DNA assembly master mix was then added, volume was 5 uL. If necessary, NFW was added to bring the final volume of the reaction mix to 10 uL. The reaction tube/plate was vortexed briefly, centrifuged for 10 seconds at 2000 G, and run on a thermocycler for 30 minutes at 65° C. Samples were cooled to 4° C. and then used immediately or stored at −80° C.

### Bacterial Strains and Transformation

NEB Stable Competent E. coli (New England Biosciences) and EPI400 (Lucigen) were used for transformation and cloning of plasmids and genomes. pUC19 was used as control DNA for transformations. Bacterial colonies were picked and individual clones taken forward with liquid culture. Luria-Berani medium, supplemented with ampicillin or kanamycin (50 mg/mL) as a selection marker, was used to grow strains at 30° C. Plasmid purification was done with QIAprep Spin Miniprep Kit (Qiagen) for downstream analysis. DNA integrity and size were analyzed by gel electrophoresis.

### Sequencing and Bioinformatics

Library preparation of purified DNA was conducted using the Rapid Barcoding kit (Oxford Nanopore Technologies). The protocol was completed according to the manufacturer's recommendations. Afterwards, samples were verified by nanopore sequencing using the MinION (Oxford Nanopore Technologies). The MinKNOW software was used for data acquisition, basecalling, and demultiplexing. The fast5/fastq files generated were then aligned to a reference sequence using the Minimap2 program. Additionally, EPI2ME was used sequence validation, whereby reads were demultiplexed by length (bp), assembled, circularized, polished to establish a consensus sequence, and annotated using Addgene and Swissprot as curated databases.

### Transfection

BHK-21 or 293T cells (ATCC) were seeded in multiwell plates, including 6, 12, 24, 48, or 96-well plates, or tissue culture flasks, including T25, T75, T175 or multi-layer cell factories, in complete media. After 24 hours, the cells were washed with PBS (1×) and transfected with the complete viral genome or plasmid using Lipofectamine 3000 (Invitrogen). Virus growth kinetics or protein expression was monitored up to 72 hours post-transfection. Viruses were then harvested, clarified, concentrated if needed, and then used immediately or stored at −80° C. until needed. Transfections using plasmids were then analyzed for protein expression.

What is claimed is:

1. A method of producing a modular viral genome comprising:
a. selecting one or more synthesized DNA fragments comprising a nucleic acid, wherein each DNA fragment comprises a first viral adaptor (VA) and a second VA, wherein the first VA is different than the second VA, and wherein each VA is located on a terminal end of the nucleic acid; and
b. assembling the one or more synthesized DNA fragments, wherein a first synthesized DNA fragment is assembled or joined to a second synthesized DNA fragment via homologous VA regions,
thereby producing a modular viral genome.

2. The method of claim 1, wherein the one or more synthesized DNA fragments comprise a gene.

3. The method of claim 1, wherein the one or more synthesized DNA fragments comprise one or more genes.

4. The method of claim 1, wherein the one or more synthesized DNA fragments comprise one or more genes from a virus.

5. The method of claim 4, wherein the virus is selected from the group consisting of measles virus (MV), rabies virus, Gibbon Ape Leukemia Virus (GALV), Sendai Virus, Seneca valley virus (SVV), adenovirus (Ad), adeno-associated viruses (AAV), herpes simplex virus (HSV), vaccinia virus (VV), vesicular stomatitis virus (VSV); autonomous parvovirus, myxoma virus (MYXV), Newcastle disease virus (NDV), reovirus, retrovirus, alphaviruses, herpesviruses, influenza virus, Sindbis virus (SINV), poxvirus, coronavirus, coronaviridae, Tobacco Mosaic Virus (TMV), Cowpea Mosaic Virus (CPMV), Semliki Forest Virus (SFV), Venezuelan equine encephalitis virus (VEEV), enterovirus, hepatitis B virus (HBV), hepatitis C virus (HCV), hepatitis D virus (HDV), and simian virus 40 (SV40).

6. The method of claim 4, wherein the virus is Vesicular stomatitis virus (VSV).

7. The method of claim 4, wherein the virus is Sindbis virus (SINV).

8. The method of claim 1, wherein the one or more synthesized DNA fragments comprise one or more genes from one or more viruses.

9. The method of claim 1, wherein a first synthesized DNA fragment comprises one or more genes from a first virus and a second synthesized DNA fragment comprises one or more genes from a second virus.

10. The method of claim 1, wherein the modular viral genome comprises at least three synthesized DNA fragments.

11. The method of claim 1, wherein the modular viral genome comprises at least four synthesized DNA fragments.

12. The method of claim 1, wherein the one or more synthesized DNA fragments comprise a coding sequence (CDS), an mRNA, a 5′ untranslated region (UTR), a 3′ UTR, a signal peptide, or a mature peptide.

13. The method of claim 1, wherein the one or more synthesized DNA fragments comprise a genetic kill switch, a reporter gene, a therapeutic agent, a watermark, a barcode, or a diagnostic agent.

14. The method of claim 1, wherein at least one synthesized DNA fragment comprises a nucleocapsid protein (N) of VSV.

15. The method of claim 1, wherein at least one synthesized DNA fragment comprises a phosphoprotein (P) of VSV.

16. The method of claim 1, wherein at least one synthesized DNA fragment comprises a matrix protein (M) of VSV.

17. The method of claim 1, wherein at least one synthesized DNA fragment comprises a glycoprotein (G) of VSV.

18. The method of claim 1, wherein at least one synthesized DNA fragment comprises a nucleocapsid protein (N), a phosphoprotein (P), and a matrix protein (M) of VSV.

19. The method of claim 1, wherein at least one synthesized DNA fragment comprises a nonstructural protein 1 (nsP1) of SINV.

20. The method of claim 1, wherein at least one synthesized DNA fragment comprises a nonstructural protein 2 (nsP2) of SINV.

21. The method of claim 1, wherein at least one synthesized DNA fragment comprises a nonstructural protein 3 (nsP3) of SINV.

22. The method of claim 1, wherein at least one synthesized DNA fragment comprises a nonstructural protein 4 (nsP4) of SINV.

23. The method of claim 1, wherein at least one synthesized DNA fragment comprises a capsid protein (C) of SINV.

24. The method of claim 1, wherein at least one synthesized DNA fragment comprises a spike glycoprotein E1 (E1) of SINV.

25. The method of claim 1, wherein at least one synthesized DNA fragment comprises a spike glycoprotein E2 (E2) of SINV.

26. The method of claim 1, wherein at least one synthesized DNA fragment comprises an assembly protein E3 (E3) of SINV.

27. The method of claim 1, wherein at least one synthesized DNA fragment comprises a nonstructural protein 1 (nsP1), a nonstructural protein 2 (nsP2), a nonstructural protein 3 (nsP3), a nonstructural protein 4 (nsP4), a capsid protein (C), a spike glycoprotein E1 (E1), a spike glycoprotein E2 (E2), and an assembly protein E3 (E3) of SINV.

28. A method of producing a modular viral genome comprising:
a. selecting a synthesized first DNA fragment comprising a nucleic acid, a first viral adaptor (VA) and a second VA on each terminal end of the nucleic acid, wherein the first VA is different than the second VA;
b. selecting a synthesized second DNA fragment comprising a nucleic acid, a first VA and a second VA on each terminal end of the nucleic acid, wherein the first VA is different than the second VA;
c. assembling the synthesized first DNA fragment and the synthesized second DNA fragment, wherein at least one VA from the synthesized first DNA fragment is homologous to at least one VA from the synthesized second DNA fragment.

29. The method of claim 28, further comprising selecting a synthesized third DNA fragment comprising a nucleic acid, a first VA and a second VA on each terminal end of the nucleic acid, wherein the first VA is different than the second VA, and assembling the synthesized third DNA fragment with the synthesized second DNA fragment, wherein at least one VA from the synthesized second DNA fragment is homologous to at least one VA from the synthesized third DNA fragment.

30. A method of producing a modular viral genome comprising:
a. designing a first DNA sequence;
b. designing a viral adaptor (VA) region for each terminal end of the first DNA sequence;
c. synthesizing a first DNA fragment, wherein the first DNA fragment comprises a first VA (VA1) on a first terminal end of a first nucleic acid and a second VA (VA2) on a second terminal end of the first nucleic acid;
d. designing a second DNA sequence;
e. designing a VA region for each terminal end of the second DNA sequence;
f. synthesizing a second DNA fragment with a second VA (VA2) on a first terminal end of a second nucleic acid and a third VA (VA3) on a second terminal end of the second nucleic acid; and
g. assembling the synthesized first DNA fragment and the synthesized second DNA fragment via the homologous VA2 regions of each DNA fragment to form a viral genome.

31. The method of claim 30, further comprising synthesizing a third DNA fragment comprising a third nucleic acid with a third VA (VA3) on a first terminal end of the third nucleic acid and a fourth VA (VA4) on a second terminal end of the third nucleic acid, and assembling the synthesized third DNA fragment to the synthesized second DNA fragment via the homologous VA3 regions.

32. The method of claim 30, wherein the first DNA fragment and/or the second DNA fragment each comprise a gene.

33. The method of claim 30, wherein the first DNA fragment and/or the second DNA fragment each comprises one or more genes.

34. The method of claim 30, wherein the first DNA fragment and/or the second DNA fragment comprises one or more genes of Vesicular stomatitis virus (VSV).

35. The method of claim 30, wherein the first DNA fragment and/or the second DNA fragment comprises one or more genes of Sindbis virus (SINV).

36. The method of claim 30, wherein the first DNA fragment comprises a nucleocapsid protein (N), a phosphoprotein (P), and a matrix protein (M) of VSV.

37. The method of claim 30, wherein the second DNA fragment comprises a glycoprotein (G) of VSV.

38. The method of claim 30, wherein the first DNA fragment and/or the second DNA fragment comprises a nonstructural protein 1 (nsP1), a nonstructural protein 2 (nsP2), a nonstructural protein 3 (nsP3), a nonstructural protein 4 (nsP4), a capsid protein (C), a spike glycoprotein E1 (E1), a spike glycoprotein E2 (E2), and an assembly protein E3 (E3) of SINV.

39. The method of claim 30, wherein the first DNA fragment and/or the second DNA fragment comprise a coding sequence (CDS), an mRNA, a 5′ untranslated region (UTR), a 3′ UTR, a signal peptide, or a mature peptide.

40. The method of claim 30, wherein the first DNA fragment and/or the second DNA fragment comprise a genetic kill switch, a reporter gene, a therapeutic agent, a watermark, a barcode, or a diagnostic agent.

41. A modular viral genome comprising one or more synthesized DNA fragments, wherein each fragment is synthesized with a viral adaptor (VA) region on each terminal end; wherein the synthesized DNA fragments are assembled via homologous VA regions of the one or more DNA fragments.
